(19) United States
(12) Patent Application Publication
(10) Pub. No.: US 2017/0041686 A1
(43) Pub. Date: Feb. 9, 2017

(54) METHOD FOR BROADCASTING SPORTS DATA INCLUDING BIO-SIGNAL AND LIVE VIDEO DATA

(71) Applicant: Samsung Electronics Co., Ltd., Suwon-si (KR)

(72) Inventors: Sang Wook PARK, Hwaseong-si (KR); Hyung Jong KOH, Seongnam-si (KR); Yong In PARK, Seoul (KR); Seung Chul SHIN, Seoul (KR); Yun Cheol HAN, Yongin-si (KR)

(21) Appl. No.: 15/230,343

(22) Filed: Aug. 5, 2016

(30) Foreign Application Priority Data
Aug. 6, 2015 (KR) 10-2015-0110873

(51) Int. Cl. H04N 21/81; A61B 5/0402; H04N 21/242; A63B 71/06; A63B 57/00; A61B 5/00; A61B 5/024

(52) U.S. Cl. CPC H04N 21/8133; A61B 5/0006; A61B 5/0022; A61B 5/0402; A61B 5/02416; A63B 71/0622; A63B 57/00; H04N 21/242

(57) ABSTRACT

A method for broadcasting sports includes receiving a first bio-signal output from a first sensor attached to a first sports participant of a sporting event through a first wireless communications module, receiving a second bio-signal output from a second sensor attached to a second sports participant of the sporting event through a second wireless communications module, receiving live video data of the first sports participant and the second sports participant through cameras, and generating sports data including at least one of the first bio-signal and the second bio-signal and the live video data.

### CROSS-REFERENCE TO RELATED APPLICATION

This application claims priority under 35 U.S.C. §119(a) from Korean Patent Application No. 10-2015-0110873 filed on Aug. 6, 2015, the disclosure of which is hereby incorporated by reference in its entirety.

### BACKGROUND

Embodiments of the present inventive concept relate to a method for broadcasting sports data, and more particularly, to a method for collecting a bio-signal of a sports participant in real time or substantially real time and broadcasting sports data including the collected bio-signal and a live video data of a sporting event performed by the sport participant.

A broadcasting system may record images of players participating in a sporting event using cameras, and broadcast the recorded images to TV sets. The broadcasting system may insert captions or advertising subtitles on the recorded images that are to be broadcast to the TV sets.

Viewers may view a sporting event through images of the event and a commentary by a commentator displayed on TV sets. However, the commentator does not know a physical condition, a mental condition, and/or a psychological condition of each of the players and the commentator may not correctly provide the viewers with current conditions of each of the players. That is, a broadcasting system of the related art may not automatically collect and provide a physical condition, a mental condition, and/or a psychological condition of each of the players participating in a sporting event.

### SUMMARY

An example embodiment of the present inventive concepts is directed to a method for broadcasting sports data, including receiving a first bio-signal output from a first sensor attached to a first sports participant through a first wireless communications module, receiving a second bio-signal output from a second sensor attached to a second sports participant through a second wireless communications module, receiving live video data of the first sports participant and the second sports participant through cameras, and generating sports data including at least one of the first bio-signal and the second bio-signal and the live video data.

Generating the sports data may generate the sports data so that the at least one of the first bio-signal and the second bio-signal is superimposed on a live video related to the live video data.

Receiving the first bio-signal, the second bio-signal and the live video data may be performed in real time or substantially real time during a sporting event.

The method for broadcasting sports data further includes broadcasting the sports data to at least one end user device, in which the at least one end user device may include a TV, a computer, or a mobile device. The broadcasting may be performed in real time or substantially real time during the sporting event. The sporting event may be a static-type sporting event or a dynamic-type sporting event. Each of the first bio-signal and the second bio-signal may be data related to electrocardiography (ECG) or data related to photoplethysmography (PPG).

When the live video data include a first video data of the first sports participant or the second sports participant, the broadcasting may broadcast the sports data including the first live video data, the first bio-signal, and the second bio-signal in real time or substantially real time during a sporting event.

The generating sports data may include reading a first previously received bio-signal of the first sports participant and a second previously received bio-signal of the second sports participant from a database, and generating the sports data including the first previously received bio-signal, the first bio-signal, the second previously received bio-signal, the second bio-signal, and the live video data.

The method for broadcasting sports data may further include broadcasting the sports data to at least one end user device through a wireless communications link in which the wireless communications link may be based on an ATSC communications standard, an ATSC-M/H communications standard, a terrestrial-digital multimedia broadcasting (T-DMB) communications standard, or a satellite-digital multimedia broadcasting (S-DMB) communications standard.

The broadcasting broadcasts the sports data to the at least one end user device through the wireless communications link in real time or substantially real time during a sporting event. The first bio-signal and the second bio-signal may be data respectively related to a heart rate of the first sports participant and the second sports participant.

An example embodiment of the present inventive concepts is directed to a method for broadcasting sports data, including receiving a first bio-signal output from a first sensor attached to a first sports participant through a first wireless communications module in real time or substantially real time, receiving a second bio-signal output from a second sensor attached to a second sports participant through a second wireless communications module in real time or substantially real time, receiving a first live video data of the first sports participant through at least one first camera recording a sporting event, processing the first bio-signal and the second bio-signal so that the first bio-signal and the second bio-signal are combined with the first live video data to generate a first sports data, and broadcasting the first sports data to at least one end user device through a wireless communications link to display the first sports data on the at least one end user device.

The method for broadcasting sports data further includes receiving a second live video data of the second sports participant through at least one second camera recording the sporting event, processing the first bio-signal and the second bio-signal so that the first bio-signal and the second bio-signal are combined with the second live video data to generate a second sports data, and broadcasting the first sports data and the second sports data to the at least one end user device through the wireless communications link to display the first sports data and the second sport data on the at least one end user device.

The first bio-signal and the second bio-signal may be data respectively related to a heart rate of the first sports participant and the second sports participant.

At least one of the first bio-signal and the second bio-signal may be superimposed on the first live video data to generate the first sports data. At least one of the first bio-signal and the second bio-signal may be superimposed on the second live video data to generate the second sports data.

An example embodiment of the present inventive concept is directed to a method to generate sports data, including receiving at a synchronization processor first current bio-signal data for a first sports participant for a current sporting event; determining at the synchronization processor whether previously received first bio-signal data for the first sports participant for a previous sporting event in which the first sports participant participated is to be displayed with the first current bio-signal data; receiving at the synchronization processor first live video data of the first sports participant participating in the current sporting event; combining the first current bio-signal data and the previously received first bio-signal data with the first live video data if it is determined that the previously received first bio-signal data is to be displayed with the first current bio-signal data; and combining the first current bio-signal data with the first live video data if it is determined that the previously received first bio-signal data is not to be displayed with the first current bio-signal data.

Combining the first current bio-signal data and the previously received first bio-signal data with the first live video data may include superimposing the first current bio-signal data and the previously received first bio-signal data on the first live video data. Similarly, combining the first current bio-signal data with the first live video data may include superimposing the first current bio-signal data on the first live video data.

In one embodiment, the previously received first bio-signal data may be an average of a plurality of previously received first bio-signal data.

The method to generate sports data may further include receiving at a synchronization processor second current bio-signal data for a second sports participant for a current sporting event; determining at the synchronization processor whether previously received second bio-signal data for the second sports participant for a previous sporting event in which the second sports participant participated is to be displayed with the second current bio-signal data; receiving at the synchronization processor second live video data of the second sports participant participating in the current sporting event; combining the second current bio-signal data and the previously received second bio-signal data with the second live video data if it is determined that the previously received second bio-signal data is to be displayed with the second current bio-signal data; and combining the second current bio-signal data with the second live video data if it is determined that the previously received second bio-signal data is not to be displayed with the second current bio-signal data.

Combining the second current bio-signal data and the previously received second bio-signal data with the second live video data may include superimposing the first current bio-signal data and the previously received first bio-signal data on the second live video data. Similarly, combining the first current bio-signal data with the second live video data comprises superimposing the first current bio-signal data on the second live video data.

In one embodiment, the previously received first bio-signal data may be an average of a plurality of previously received first bio-signal data.

In one embodiment, the first bio-signal data and the second bio-signal data may be data respectively related to a heart rate of the first sports participant and the second sports participant.

### BRIEF DESCRIPTION OF THE DRAWINGS

These and/or other aspects and advantages of the present inventive concepts will become apparent and more readily appreciated from the following description of the embodiments, taken in conjunction with the accompanying drawings of which:

FIG. 1 is a block diagram that shows a communications network system according to an exemplary embodiment of the present inventive concepts;

FIG. 2 is a block diagram of a broadcasting system portion of the communications network system shown in FIG. 1;

FIG. 3 is a flowchart that depicting processes of broadcasting sports data using the communications network system shown in FIG. 1;

FIGS. 4A and 4B are drawings that depict a process of generating sports data including bio-signals and live video data and a process of displaying the sports data on a display device according to embodiments of the present inventive concepts;

FIGS. 5A and 5B are drawings that depict the process of generating sports data including bio-signals and live video data and the process of displaying the sports data in a display device according to embodiments of the present inventive concepts;

FIGS. 6A and 6B are drawings that depict the process of generating sports data including bio-signals and live video data and the process of displaying the sports data in a display device according to embodiments of the present inventive concepts;

FIG. 7 is a flowchart that describes a process of generating sports data according to an embodiment.

### DETAILED DESCRIPTION OF THE EMBODIMENTS

The present inventive concepts now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity. Like numbers refer to like elements throughout.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. As used herein, the term “and/or” includes any and all combinations of one or more of the associated listed items and may be abbreviated as “/.”

It will be understood that, although the terms first, second, etc. may be used herein to describe various elements, these elements should not be limited by these terms. These terms are only used to distinguish one element from another. For example, a first signal could be termed a second signal, and, similarly, a second signal could be termed a first signal without departing from the teachings of the disclosure.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and/or the present application, and will not be interpreted in an idealized or overly formal sense unless expressly so defined herein.

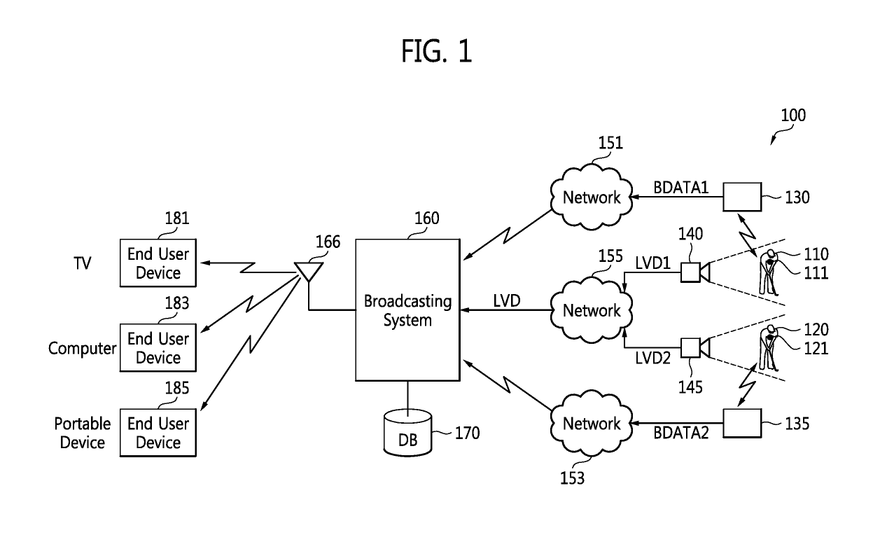

FIG. 1 is a block diagram that shows a communications network system 100 according to some embodiments of the present inventive concepts. Referring to FIG. 1, a communications network system 100 may include a first sensor 111 attached to a first sports participant 110, a second sensor 121 attached to a second sports participant 120, a first wireless communications module 130, a second wireless communications module 135, a first camera 140, a second camera 145, networks 151, 153, and 155, a broadcasting system 160, and a plurality of end user devices 181, 183, and 185. The communications network system 100 may further include an antenna 166 that can broadcast sports data generated by the broadcasting system 160 and a database 170 that may store the sports data.

Each of the sports participants 110 and 120 may be a participant or a player in a static-type sporting event or a dynamic-type sporting event. A static-type sporting event may be static sports match (or game) and includes golf games, archery games, chess, and the like; however, it is not limited thereto. That is, a static-type sporting event may be a sporting event in which the participants are relatively physically inactive. A dynamic-type sporting event may be a dynamic sports match (or game), and includes American Football games, baseball games, basketball games, volleyball games, ice hockey games, and the like; however, it is not limited thereto. That is, a dynamic-type sporting event may be a sporting event in which the participants are relatively physically active.

The first sensor 111 may be one or more sensors that may sense a first bio-signal (a first biological-signal, or a first bio-data) of the first sports participant 110. For example, the first sensor 111 may be a sensor that can sense electrocardiography (ECG) or photoplethysmography (PPG) of the first sports participant 110; however, it is not limited thereto. When the first sensor 111 generates a signal related to a heart rate as a bio-signal, the first sensor 111 may be attached to the first sports participant 110 at a position (for example, around a heart or a wrist; however, it is not limited thereto) that is capable of sensing a heart rate of the first sports participant 110.

The second sensor 121 may be one or more sensors that can sense a second bio-signal (a second biological-signal or a second bio-data) of the second sports participant 120. For example, the second sensor 121 may be a sensor that can sense ECG or PPG of the second sports participant 120; however, it is not limited thereto. When the second sensor 121 generates a signal related to a heart rate as bio-signal, the second sensor 121 may be attached to the second sports participant 120 at a position (for example, around a heart or a wrist; however, it is not limited thereto) that is capable of sensing a heart rate of the second sports participant 120.

Each of the sensors 111 and 121 may perform a function of sensing one or more bio-signals of each of the sports participants 110 and 120 and a function of respectively transmitting the one or more sensed bio-signals to the respective wireless communications modules 130 and 135 through a wireless communications link. If each of the sensors 111 and 121 is an ECG sensor or a PPG sensor, each of the sensors 111 and 121 may further analyze an ECG waveform or a PPG waveform of each of the sports participants 110 and 120, predict cardiac arrhythmia of each of the sports participants 110 and 120 according to a result of the analysis, and output a result of the prediction through an output device (for example, a speaker) included in each of the sensors 111 and 121 and/or output the result of the prediction to each respective wireless communications modules 130 and 135.

Each of the sensors 111 and 121 may be embodied as an Internet of Things (IoT) device or an Internet of Everything (IoE) device. According to some exemplary embodiments, a wireless communications link may be established using Bluetooth, Bluetooth low energy (BLE), near-field communication (NFC), Zigbee, and/or radio frequency identification (RFID); however, it is not limited thereto.

The first wireless communications module 130 may receive a first bio-signal (BDATA1) of the first sports participant 110 that has been output from the first sensor 111 and transmit the first bio-signal BDATA1 to a first network 151. The first wireless communications module 130, which can perform wireless communications with the first sensor 111, may be a smart phone, an IoT device, an IoE device, and/or a wearable device; however, it is not limited thereto.

The second wireless communications module 135 may receive a second bio-signal BDATA2 of the second sports participant 120 that has been output from the second sensor 121, and transmit the second bio-signal BDATA2 to a second network 153. The second wireless communications module 135, which can perform wireless communications with the second sensor 121, may be a smart phone, an IoT device, an IoE device, and/or a wearable device; however, it is not limited thereto.

The first camera 140 that records a sporting event may generate a first live video data LVD1 of the first sports participant 110 and transmit the first live video data LVD1 to a third network 155. The second camera 145 that records the sporting event may generate a second live video data LVD2 of the second sports participant 120 and transmit the second live video data LVD2 to the third network 155. Each of the cameras 140 and 145 may be embodied as an automatic video camera that can automatically track each of the sports participants 110 and 120; however, it is not limited thereto.

Each of the networks 151, 153, and 155 may be a network that uses the same communications method or, alternatively, a different communications method. According to embodiments, each of the networks 151, 153, and 155 may be a local area network (LAN), a wireless local area network (WLAN), such as wireless fidelity (Wi-Fi), a wireless personal area network (WPAN), a wireless universal serial bus (USB), a 3rd generation (3G) mobile communications network, a 4^{th} generation (4G) mobile communications network, a long term evolution (LTE™) mobile communications network, the Internet, or the wireless Internet; however, it is not limited thereto.

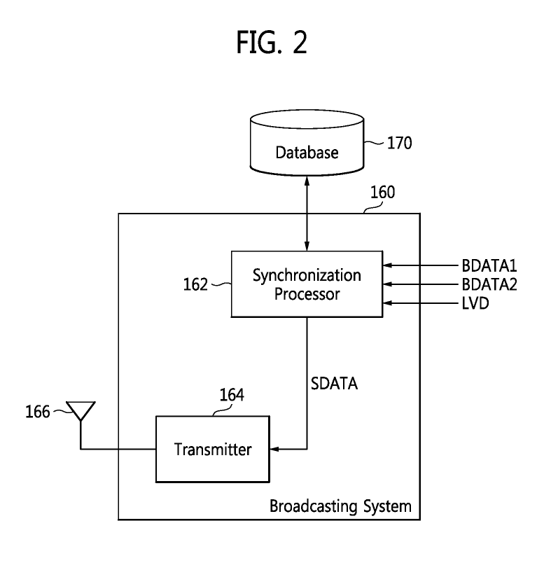

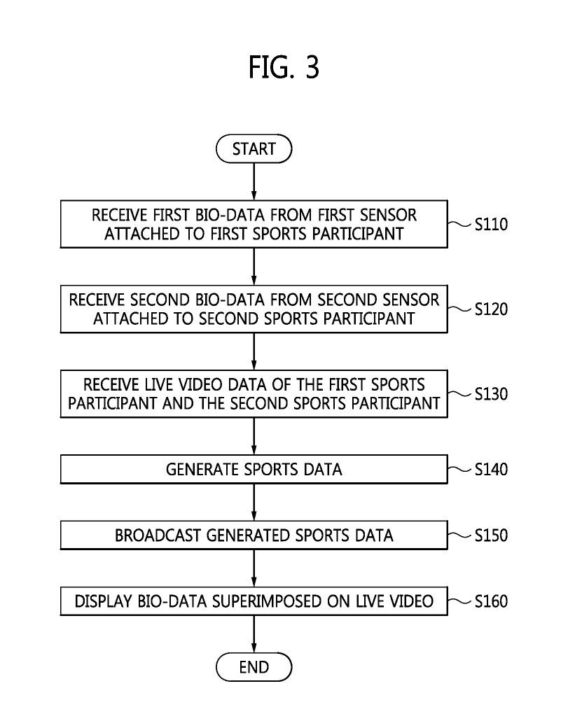

FIG. 2 is a block diagram of a broadcasting system portion of the communications system network shown in FIG. 1, and FIG. 3 is a flowchart that describes processes of broadcasting sports data using the communications network system shown in FIG. 1.

Referring to FIGS. 1 to 3, the broadcasting system 160 may receive the first bio-signal BDATA1 of the first sports participant 110 through the first network 151 (S110). For example, a synchronization processor 162 of the broadcasting system 160 may receive the first bio-signal BDATA1 output from the first sensor 111 attached to the first sports participant 110 through the first wireless communication module 130 and the first network 151 (S110).

The broadcasting system 160 may receive a second bio-signal BDATA2 of the second sports participant 120 through the second network 153 (S120). For example, the synchronization processor 162 of the broadcasting system 160 may receive the second bio-signal BDATA2 output from the second sensor 121 attached to the second sports participant 120 through the second wireless communication module 135 and the second network 152 (S120).

Even though it is shown in FIG. 3 that a process (S110) of receiving the first bio-signal BDATA1 is performed prior to a process (S120) of receiving the second bio-signal BDATA2, the process (S110) of receiving the first bio-signal BDATA1 and the process (S120) of receiving the second bio-signal BDATA2 may be performed simultaneously or substantially simultaneously, in a parallel manner, may be overlapped in time with respect to each other, or the process (S110) of receiving the first bio-signal BDATA1 may be performed later than the process (S120) of receiving the second bio-signal BDATA2.

The broadcasting system 160 may receive at least one of the first live video data LVD1 of the first sports participant 110 and the second live video data LVD2 of the second sports participant 120 through the third network 155 (S130). Live video data LVD may include at least one of the first live video data LVD1 and the second live video data LVD2. For example, each of the live video data LVD1 and LVD2 output from each of the cameras 140 and 145 may be transmitted to the synchronization processor 162 of the broadcasting system 160 through the third network 155 (S130).

The broadcasting system 160 may generate sports data including at least one of the first bio-signal BDATA1 and the second bio-signal BDATA2 and at least one of the first live video data LVD1 and the second live video data LVD2 (S140).

For example, the synchronization processor 162 of the broadcasting system 160 may process at least one of the first bio-signal BDATA1 and the second bio-signal BDATA2 so that at least one of the first bio-signal BDATA1 and the second bio-signal BDATA2 is superimposed on or onto a video (or a live video) corresponding to at least one of the first live video data LVD1 and the second live video data LVD2, and generate sports data SDATA corresponding to a result of the processing (S140).

The synchronization processor 162 of the broadcasting system 160 may store the first bio-signal (or data) BDATA1, the second bio-signal (or data) BDATA2, the first live video data LVD1, the second live video data LVD2, and/or the sports data SDATA in a database 170. The database 170 may store a previously received bio-signal of the first sports participant 110, a previously received live video data of the first sports participant 110, a previously received bio-signal of the second sports participant 120, and/or a previously received live video data of the second sports participant 120.

A transmitter 164 of the broadcasting system 160 may broadcast the sports data SDATA to a plurality of end user devices 181, 183, and 185 through the antenna 166 under the control of the synchronization processor 162 (S150).

According to some exemplary embodiments, the broadcasting system 160 may generate sports data SDATA based on an advanced television system committee (ATSC) standard and/or an ATSC-mobile/handheld (ATSC-M/H) standard, and broadcast the sports data SDATA. According to some exemplary embodiments, the broadcasting system 160 may generate sports data SDATA based on a digital video broadcasting-cable (DVB-C) standard, a digital video broadcasting-terrestrial (DVB-T) standard, a DVB-satellite (DVB-S) standard, a DVB-satellite services to handhelds (DVB-SH) standard, and/or broadcast the sports data SDATA.

According to some exemplary embodiments, the broadcasting system 160 may generate sports data SDATA based on a China Digital Multimedia Broadcast-Terrestrial/Handheld (CDMB-T/H) standard and/or a China Mobile Multimedia Broadcasting (CMMB) standard and broadcast the sports data SDATA.

According to some exemplary embodiments, the broadcasting system 160 may generate sports data SDATA based on an Integrated Services Digital Broadcasting (ISDB) standard, an ISDB-satellite (ISDB-S) standard, an ISDB-terrestrial (ISDB-T) standard, an ISDB-cable (ISDB-C) standard, a 1 seg standard, an international system for digital broadcast, terrestrial, and/or Brazilian version (ISDB-Tb) standard, and broadcast the sports data SDATA.

According to some exemplary embodiments, the broadcasting system 160 may generate sports data SDATA based on a national television system committee (NTSC) standard, a phase alternating line (PAL) standard, and/or a sequential color with memory (SECAM) standard, and broadcast the sports data SDATA. According to exemplary embodiments, the broadcasting system 160 may generate sports data SDATA based on a terrestrial-digital multimedia broadcasting (T-DMB) standard and/or a satellite-digital multimedia broadcasting (S-DMB) standard, and broadcast the sports data SDATA.

Each of the user end devices 181, 183, and 185 may receive sports data SDATA and display live video including at least one of the bio-signal BDATA1 and BDATA2 on a display device of each of the end user devices 181, 183, and 185 (S160). For example, a first end user device 181 may be a TV (or a digital TV), a second end user device 183 may be a computer, and a third end user device 185 may be a portable device; however, these are not limited thereto. The portable device may be a laptop computer, a mobile phone, a smart phone, a tablet PC, a personal digital assistant (PDA), an enterprise digital assistant (EDA), a portable multimedia player (PMP), a mobile internet device (MID), a wearable computer, an IoT device, or an IoE device; however, it is not limited thereto.

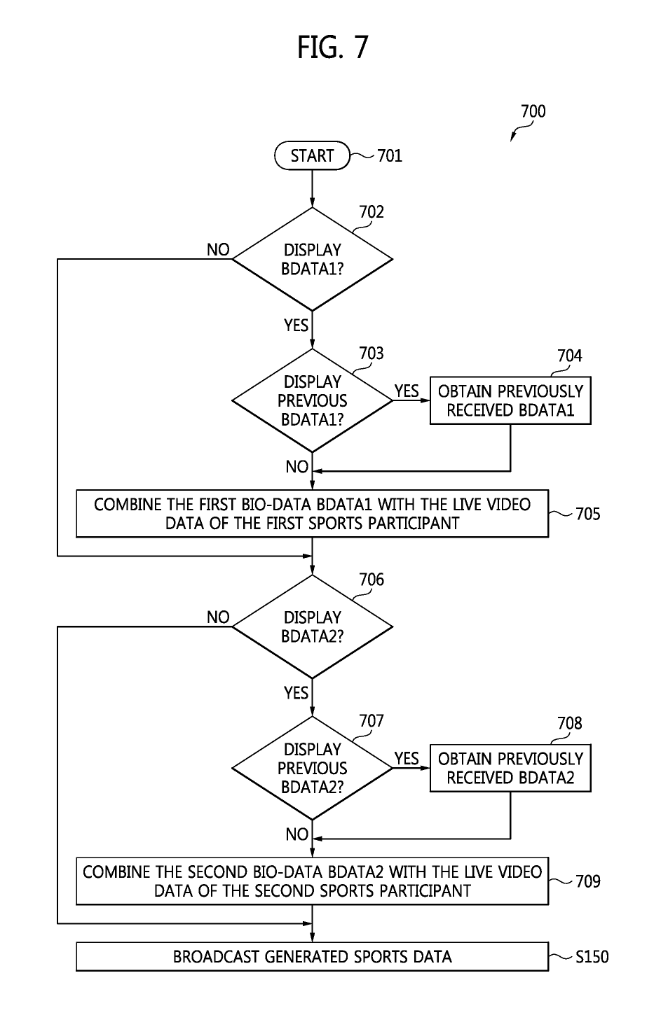

FIG. 7 is a flowchart that describes a process of generating sports data 700 according to an embodiment. In particular, FIG. 7 provides details regarding generating sports data S140 shown in FIG. 3. The process 700 begins at operation 701. At operation 702, it is determined whether the synchronization processor 162 is to generate sports data that includes the first bio-signal data BDATA1 for the first sports participant. If, at operation 701, the synchronization processor 162 is to generate sports data that includes the first bio-signal data BDATA1, flow continues to operation 703, wherein it is determined whether the synchronization processor 162 is to obtain and display previously received first bio-signal data BDATA1 for the first sports participant. If so, flow continues to operation 704, where the synchronization processor 162 accesses the database 170 to obtain previously received first bio-signal data BDATA1, and flow continues to operation 705. If, at operation 703, the synchronization processor 162 is not to obtain and display previously received first bio-signal data BDATA1 for the first sports participant, flow continues to operation 705. At operation 705, the synchronization processor 162 combines the first bio-signal data BDATA1 into the live video data for the first sports participant. In one embodiment, the first bio-signal data BDATA1 is superimposed on the live video data for the first sports participant. Flow continues to operation 706.

At operation 706, it is determined whether the synchronization processor 162 is to generate sports data that includes the second bio-signal data BDATA2 for the second sports participant. If, at operation 706, the synchronization processor 162 is to generate sports data that includes the second bio-signal data BDATA2, flow continues to operation 707, wherein it is determined whether the synchronization processor 162 is to obtain and display previously received second bio-signal data BDATA2 for the second sports participant. If so, flow continues to operation 708, where the synchronization processor 162 accesses the database 170 to obtain previously received second bio-signal data BDATA2, and flow continues to operation 709. If, at operation 707, the synchronization processor 162 is not to obtain and display previously received second bio-signal data BDATA2 for the second sports participant, flow continues to operation 709. At operation 709, the synchronization processor 162 combines the second bio-signal data BDATA2 into the live video data for the second sports participant. In one embodiment, the second bio-signal data BDATA2 is superimposed on the live video data for the second sports participant. Flow continues to operation S150, wherein the generated sports data is broadcast.

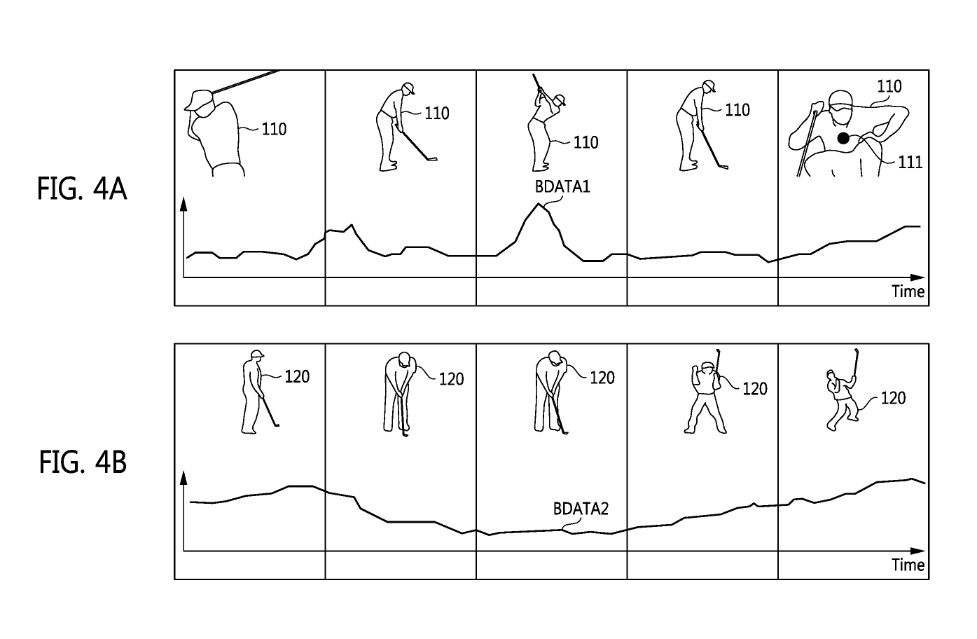

FIGS. 4A and 4B are drawings that depict a process of generating sports data including bio-signals and live video data and a process of displaying the sports data on a display device according to some exemplary embodiments of the present inventive concepts. It is shown that each of the sports participants 110 and 120 is a golfer as an exemplary embodiment in FIGS. 4A to 6B; however, a technical concept of the present inventive concepts may be applied to participants of various sporting events (or sports games).

Referring to FIGS. 1 to 4B, the synchronization processor 162 may receive the first bio-signal BDATA1 and the first live video data LVD1 in real time or substantially real time, and generate sports data SDATA including the first bio-signal BDATA1 and the first live video data LVD1 in real time or substantially real time.

The synchronization processor 162 may receive the second bio-signal VDATA2 and the second live video data LVD2 in real time or substantially real time and generate sports data SDATA including the second bio-signal BDATA2 and the second live video data LVD2 in real time or substantially real time.

The synchronization processor 162 may receive the first bio-signal BDATA1, the second bio-signal VDATA2, and the first live video data LVD1 in real time or substantially real time, and generate sports data SDATA including the first bio-signal BDATA1, the second bio-signal BDATA2, and the first live video data LVD1 in real time or substantially real time.

The synchronization processor 162 may receive the first bio-signal BDATA1, the second bio-signal BDATA2, and the second video data LVD2 in real time or substantially real time, and generate sports data SDATA including the first bio-signal BDATA1, the second bio-signal BDATA2, and the second video data LVD2 in real time or substantially real time.

The synchronization processor 162 may receive the first bio-signal BDATA1, the second bio-signal BDATA2, the first live video data LVD1, and the second video data LVD2 in real time or substantially real time, and generate sports data SDATA including the first bio-signal BDATA1, the second bio-signal BDATA2, the first live video data LVD1, and the second video data LVD2 in real time or substantially real time.

The synchronization processor 162 of FIG. 4A may generate sports data SDATA including the first bio-signal BDATA1 of the first sports participant 110 and the first live video data LVD1 of the first sports participant 110 in real time or substantially real time, and broadcast the sports data SDATA to each of the end user devices 181, 183, and 185. Each of the end user devices 181, 183, and 185 may display live video including the first bio-signal BDATA1 on a display device. Here, the live video may be a video corresponding to the first live video data LVD1. The first bio-signal BDATA1 may be displayed in different sizes at different positions in the live video under selectable control of the synchronization processor 162.

The synchronization processor 162 of FIG. 4B may generate sports data SDATA including the second bio-signal BDATA2 of the second sports participant 120 and the second live video data LVD2 of the second sports participant 120 in real time or substantially real time, and broadcast the sports data SDATA to each of the end user devices 181, 183, and 185. Each of the end user devices 181, 183, and 185 may display live video including the second bio-signal BDATA2 on a display device. Here, the live video may be a video corresponding to the second live video data LVD2. The second bio-signal BDATA2 may be displayed in different sizes at different positions in the live video under selectable control of the synchronization processor 162.

Even though it is shown in FIGS. 4A and 4B that each of the bio-signal BDATA1 and BDATA2 itself is displayed on a display of each of the end user devices 181, 183, and 185; however, it is only exemplification, and each of the bio-signal BDATA1 and BDATA2 displayed on the display may be understood as a concept including a graphics user interface GUI and/or an icon for displaying each of the bio-signal BDATA1 and BDATA2.

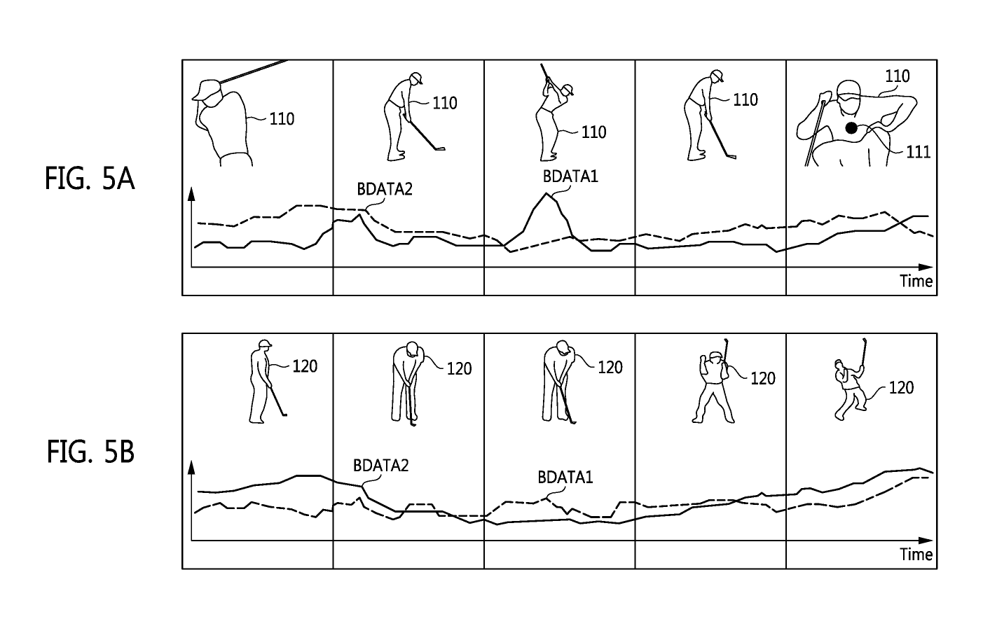

FIGS. 5A and 5B are drawings that depict the process of generating sports data including bio-signals and live video data and the process of displaying the sports data in a display device according to some exemplary embodiments of the present inventive concepts.

As shown in FIG. 5A, the synchronization processor 162 may generate sports data SDATA including the first bio-signal BDATA1 of the first sports participant 110, the second bio-signal BDATA2 of the second sports participant 120, and the first live video data LVD1 of the first sports participant 110 in real time or substantially real time, and broadcast the sports data SDATA to each of end user devices 181, 183, and 185.

Each of the end user devices 181, 183, and 185 may display a live video including the first bio-signal BDATA1 and the second bio-signal BDATA2 on a display device. Here, the live video may be a video corresponding to the first live video data LVD1. The first bio-signal BDATA1 and the second bio-signal BDATA2 may be displayed in different sizes at different positions in the live video under selectable control of the synchronization processor 162 and/or the user of the end user device 181, 183 and 185.

As shown in FIG. 5A, when the first sports participant 110 continues with a current play, the second sports participant 120 may not be continuing with the current play. The first bio-signal BDATA1 may be data (or signal) related to a heart rate of the first sports participant 110 continuing with the current play, and the second bio-signal BDATA2 may be data (or signal) related to a heart rate of the second sports participant 120 that is not continuing with the current play. Accordingly, a viewer can view not only the heart rate of the first sports participant 110, but also the heart rate of the second sports participant 120 while viewing a video corresponding to the first live video data LVD1.

As shown in FIG. 5B, the synchronization processor 162 may generate sports data SDATA including the first bio-signal BDATA1 of the first sports participant 110, the second bio-signal BDATA2 of the second sports participant 120, and the second live video data LVD2 of the second sports participant 120 in real time or substantially real time, and broadcast the sports data SDATA to each of the end user devices 181, 183, and 185.

Each of the end user devices 181, 183, 185 may display a live video including the first bio-signal BDATA1 and the second bio-signal BDATA2 on a display device. Here, the live video may be a video corresponding to the second live video data LVD2. The first bio-signal BDATA1 and the second bio-signal BDATA2 may be displayed in different sizes at different positions in the live video under selectable control of the synchronization processor 162 and/or the user of the end user device 181, 183 and 185.

As shown in FIG. 5B, when the second sports participant 120 continues with a current play, the first sports participant 110 may not be continuing with the current play. The second bio-signal BDATA2 may be data related to a heart rate of the second sports participant 120 continuing with the current play, and the first bio-signal BDATA1 may be data related to a heart rate of the first sports participant 110 not that is continuing with the current play. Accordingly, a viewer can view not only the heart rate of the first sports participant 110, but also the heart rate of the second sports participant 120 while viewing a video corresponding to the second live video data LVD2.

Each of the bio-signals BDATA1 and BDATA2 displayed on a display of each of the end user devices 181, 183, and 185 may be understood as a concept including a graphics user interface (GUI) and/or an icon for displaying each of the bio-signals BDATA1 and BDATA2.

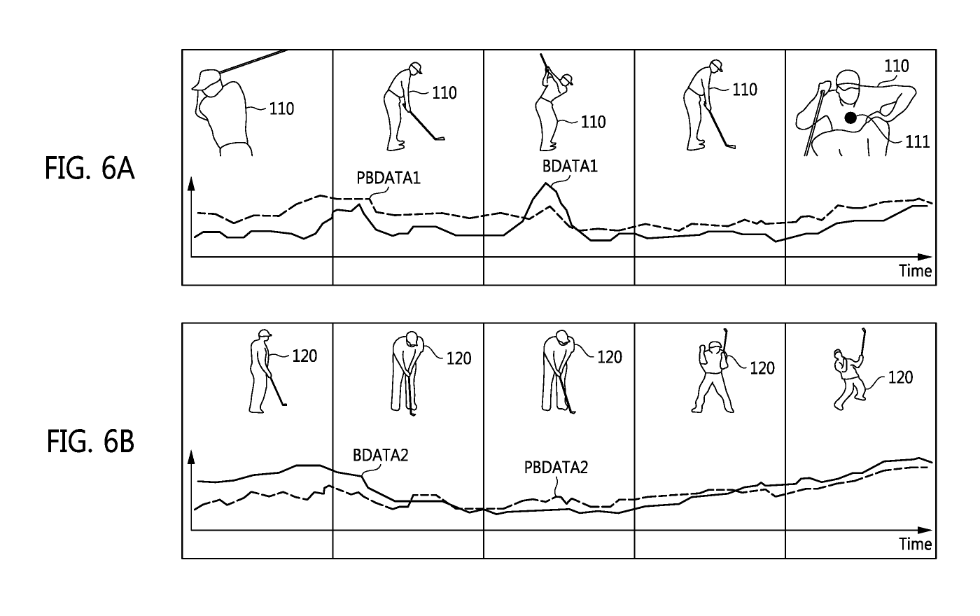

FIGS. 6A and 6B are drawings that depict the process of generating sports data including bio-signals and live video data and the process of displaying the sports data in a display device according to some exemplary embodiments of the present inventive concepts.

As shown in FIG. 6A, the synchronization processor 162 may generate sports data SDATA including the first bio-signal BDATA1 of the first sports participant 110, a previously received bio-signal PBDATA1 of the first sports participant 110, and a first live video data LVD1 of the first sports participant 110 in real time or substantially real time, and broadcast the sports data SDATA to each of end user devices 181, 183, and 185. The previously received bio-signal PBDATA1 may be bio-signal collected by the first sensor 111 from one or more sporting events in which the first sports participant 110 previously participated. The previous bio-signal PBDATA1 may be an average value of a number of previously received bio-signals PBDATA1; however, it is not limited thereto.

Each of the end user devices 181, 183, and 185 may display live video including the first bio-signal BDATA1 and the previously received bio-signal PBDATA1 on a display device. Here, the live video may be a video corresponding to the first live video data LVD1. The first bio-signal BDATA1 and the previous bio-signal PBDATA1 may be displayed in different sizes at different positions in the live video under selectable control of the synchronization processor 162 and/or the user of the end user device 181, 183 and 185.

As shown in FIG. 6A, the first bio-signal BDATA1 may be data related to a change in a heart rate of the first sports participant 110 continuing with a current play, and the previous bio-signal PBDATA1 may be data related to a change in a heart rate of the first sports participant 110 as the first sports participant 110 participated in a previous sporting event. Accordingly, a viewer can view not only the change in the heart rate of the first sports participant 110 proceeding with the current sports, but also the change in the heart rate of the first sports participant 110 who performed in a previous sporting event while viewing a video corresponding to the first live video data LVD1.

As shown in FIG. 6B, the synchronization processor 162 may generate sports data SDATA including the second bio-signal BDATA2 of the second sports participant 120, a previously received bio-signal PBDATA2 of the second sports participant 120, and the second live video data LVD2 of the second sports participant 120 in real time or substantially real time, and broadcast the sports data SDATA to each of the end user devices 181, 183, and 185. The previously received bio-signal PBDATA2 may be bio-signal collected by the second sensor 121 from one or more sporting events in which the second sports participant 120 previously participated. The previous bio-signal PBDATA2 may be an average value of a number of previously received bio-signals PBDATA2; however, it is not limited thereto.

Each of the end user devices 181, 183, and 185 may display a live video including the second bio-signal BDATA2 and the previously received bio-signal PBDATA2 on a display device. Here, the live video may be a video corresponding to the second live video data LVD2. The second bio-signal BDATA2 and the previously received bio-signal PBDATA2 may be displayed in different sizes at different positions in the live video under selectable control of the synchronization processor 162 and/or the user of the end user device 181, 183 and 185.

As shown in FIG. 6B, the second bio-signal BDATA2 may be data related to a change in a heart rate of the second sports participant 120 continuing with a current play, and the previously received bio-signal PBDATA2 may be data related to a change in a heart rate of the second sports participant 120 as the second sports participant 120 participated in a previous sporting event. Accordingly, a viewer can view not only a change in the heart rate of the second sports participant 120 proceeding with the current sporting event, but also a change in the heart rate of the second sports participant 120 who performed in a previous sporting event while viewing a video corresponding to the second live video data LVD2.

A method for broadcasting sports data according to some embodiments of the present inventive concepts may collect bio-signal, e.g., a heart rate, of a sports participant, in real time or substantially real time, and broadcast the sports data including collected bio-signal and live video data of a sporting event.

According to the method for broadcasting sports data, a broadcasting company can broadcast tension and/or a psychological state of a sports participant to a viewer in real time or substantially real time using a change in bio-signal, e.g., a heart rate, of the sports participant. Moreover, according to the method for broadcasting sports data, a viewer can observe how a sports participant overcomes tension or a psychological state. The sports data broadcast by a broadcasting company can stimulate curiosity of a viewer on how a sports participant overcomes tension or a psychological state and intensify the broadcast of a sporting event.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A method for broadcasting sports data, the method comprising:
receiving a first bio-signal output from a first sensor attached to a first sports participant through a first wireless communications module;
receiving a second bio-signal output from a second sensor attached to a second sports participant through a second wireless communications module;
receiving live video data of the first sports participant and the second sports participant through cameras, and
generating sports data so that at least one of the first bio-signal and the second bio-signal is superimposed on a live video related to the live video data,
wherein receiving the first bio-signal, the second bio-signal and the live video data are performed in real time or substantially real time during a sporting event.

2. The method of claim 1, further comprising broadcasting the sports data to at least one end user device,
wherein the at least one end user device includes a TV, a computer, or a mobile device.

3. The method of claim 2, wherein the broadcasting is performed in real time or substantially real time during the sporting event.

4. The method of claim 3, wherein the sporting event is a static-type sporting event or a dynamic-type sporting event.

5. The method of claim 2, wherein each of the first bio-signal and the second bio-signal may be data related to electrocardiography (ECG) or data related to photoplethysmography (PPG).

6. The method of claim 5, wherein if the live video data includes a first live video data of the first sports participant or the second sports participant,
the broadcasting broadcasts the sports data including the first live video data, the first bio-signal, and the second bio-signal in real time or substantially real time during a sporting event.

7. The method of claim 1, wherein the generating sports data includes:
reading a first previously received bio-signal of the first sports participant and a second previously received bio-signal of the second sports participant from a database, and
generating the sports data including the first previously received bio-signal, the first bio-signal, the second previously received bio-signal, and the second bio-signal, and the live video data.

8. The method of claim 7, further comprising broadcasting the sports data to a at least one end user device through a wireless communications link,
wherein the wireless communications link is based on an ATSC communications standard, an ATSC-M/H communications standard, a terrestrial-digital multimedia broadcasting (T-DMB) communications standard, or a satellite-digital multimedia broadcasting (S-DMB) communications standard.

9. The method of claim 8, wherein the broadcasting broadcasts the sports data to the plurality of end user devices through the wireless communications link in real time or substantially real time during a sporting event.

10. The method of claim 1, wherein the first bio-signal and the second bio-signal is data respectively related to a heart rate of the first sports participant and the second sports participant.

11. A method for broadcasting sports data, the method comprising:
receiving a first bio-signal output from a first sensor attached to a first sports participant of a sporting event through a first wireless communications module in real time or substantially real time;
receiving a second bio-signal output from a second sensor attached to a second sports participant of a sporting event through a second wireless communications module in real time or substantially real time;
receiving a first live video data of the first sports participant through at least one first camera recording the sporting event;
processing the first bio-signal and the second bio-signal so that the first bio-signal and the second bio-signal are combined with the first live video data to generate a first sports data, and
broadcasting the first sports data to at least one end user device through a wireless communications link to display the first sports data on the at least one end user device.

12. The method of claim 11, further comprising:
receiving a second live video data of the second sports participant through at least one second camera recording the sporting event;
processing the first bio-signal and the second bio-signal so that the first bio-signal and the second bio-signal are combined with the second live video data to generate a second sports data, and
broadcasting the first sports data and the second sports data to the at least one end user device through the wireless communications link to display the first sports data and the second sports data on the at least one end user device.

13. The method of claim 11, wherein the first bio-signal and the second bio-signal is data respectively related to a heart rate of the first sports participant and the second sports participant.

14. The method of claim 11, wherein each of the first bio-signal and the second bio-signal is data related to electrocardiography (ECG) or data related to photoplethysmography (PPG).

15. The method of claim 11, wherein the wireless communications link is based on an ATSC communications standard, an ATSC-M/H communications standard, a terrestrial-digital multimedia broadcasting (T-DMB) communications standard, or a satellite-digital multimedia broadcasting (S-DMB) communications standard.

16-18. (canceled)

19. A method to generate sports data, the method comprising:
receiving at a synchronization processor first current bio-signal data for a first sports participant for a current sporting event;
determining at the synchronization processor whether previously received first bio-signal data for the first sports participant for a previous sporting event in which the first sports participant participated is to be displayed with the first current bio-signal data;
receiving at the synchronization processor first live video data of the first sports participant participating in the current sporting event;
combining the first current bio-signal data and the previously received first bio-signal data with the first live video data if it is determined that the previously received first bio-signal data is to be displayed with the first current bio-signal data; and
combining the first current bio-signal data with the first live video data if it is determined that the previously received first bio-signal data is not to be displayed with the first current bio-signal data.

20. The method of claim 19, wherein combining the first current bio-signal data and the previously received first bio-signal data with the first live video data comprises superimposing the first current bio-signal data and the previously received first bio-signal data on the first live video data.

21. The method of claim 19, wherein combining the first current bio-signal data with the first live video data comprises superimposing the first current bio-signal data on the first live video data.

22. The method of claim 19, wherein the previously received first bio-signal data is an average of a plurality of previously received first bio-signal data.

23. The method of claim 19, further comprising:
receiving at a synchronization processor second current bio-signal data for a second sports participant for a current sporting event;
determining at the synchronization processor whether previously received second bio-signal data for the second sports participant for a previous sporting event in which the second sports participant participated is to be displayed with the second current bio-signal data;
receiving at the synchronization processor second live video data of the second sports participant participating in the current sporting event;
combining the second current bio-signal data and the previously received second bio-signal data with the second live video data if it is determined that the previously received second bio-signal data is to be displayed with the second current bio-signal data; and
combining the second current bio-signal data with the second live video data if it is determined that the previously received second bio-signal data is not to be displayed with the second current bio-signal data.

24-27. (canceled)
